SEQUENCE LISTING (12 sequences)

SEQ ID NO: 1 - protein, 119 aa, Artificial
  LC5002-002 VH gamma/heavy chain variable domain
EVQLLESGGGLVQPGGSLRLSCAASGFTFNIYAMNWVRQAPGKGLEWVSVISGRGITTYYADSVKGRFTISRDNSKNTLYLQMNSLRADDTAVYYCAKGSSSWTDFDYWGQGTLVTVSS

SEQ ID NO: 2 - protein, 107 aa, Artificial
  LC5002-002 VL kappa light/chain variable domain
DIQMTQSPSSLSASVGDRVTITCRASQGISRWVAWYQQKPEKAPKSLIYAASSLQSGVPSRFSGSGSGTDFTLTISSLQPEDFATYYCQQYNSYPWTFGQGTKVEIK

SEQ ID NO: 3 - protein, 119 aa, Artificial
  LC5002-003 VH gamma/heavy chain variable domain
EVQLLESGGDLIQPGGSLRLSCAASGFTFNIYAMNWVRQAPGKGLEWVSVISGRGITTYYADSVKGRFTISRDNSKNTLYLQMDSLRAEDTAVYYCAKGSSYWTDFDYWGQGTLVTVSS

SEQ ID NO: 4 - protein, 107 aa, Artificial
  LC5002-003 VL kappa/light chain variable domain
DIQMTQSPSSLSASVGDRVTITCRASQGISSWLAWYQQKPEKAPKSLIYAASSLQSGVPSRFSGSGSGTDFTLTISSLQPEDFATYYCQQYNSYPWTFGQGTKVEIK

SEQ ID NO: 5 - protein, 118 aa, Artificial
  LC5002-005 VH gamma/heavy chain variable domain
EVQVLDSGGGLVQPGGSLRLSCTASGFTFRLYTMSWVRQTPGRGLEWVSGISGSGLSTYFADSVKGRFTISRDNSKNTVYLQMNSLRAEDTAVYYCAKEGDWIYFDSWGQGTLVIVSS

SEQ ID NO: 6 - protein, 107 aa, Artificial
  LC5002-005 VL kappa/light chain variable domain
DIQMTQSPSSLSASVGDRVTITCRASQGISSWLAWYQQKPEKAPKSLIYAASSLQSGVPSRFSGSGSGTDFTLTISSLQPEDFATYYCQQYNSHPPTFGQGTKLEIK

SEQ ID NO: 7 - protein, 123 aa, Artificial
  LC5002-007 VH gamma/heavy chain variable domain
QVQLVQSGAEVKKPGSSVKVSCKVSGGTFSSYAFSWVRQAPGQGLEWMGRIIPILGRTNYAQKFQGRVTITADKSTSTAYMEVSSLRSEDTAVYYCAREGETLDYFYYGMDVWGQGTTVTVSS

SEQ ID NO: 8 - protein, 107 aa, Artificial
  LC5002-007 VL kappa/light chain variable domain
EIVLTQSPGTLSLSPGERATLSCRASQSVSSSYLAWYQQKPGQAPRLLIYGASSRAIGIPDRFSGSGSGTDFTLTISRLEPEDFAVYYCQHYGSSLTFGGGTKVEIK

SEQ ID NO: 9 - protein, 119 aa, Artificial
  LC5002-018 VH gamma/heavy chain variable domain
EVQLLESGGGLVQPGGSLRLSCAASGFTFNIYAMNWVRQAPGKGLEWVSVISGSGVTTYYADSVKGRFTISRDNSKNTLYLQMNSLRAEDTAVYYCAKGSSWYVDFDYWGQGTLVTVSS

SEQ ID NO: 10 - protein, 107 aa, Artificial; identical to SEQ ID NO: 4
  LC5002-018 VL kappa/light chain variable domain

SEQ ID NO: 11 - protein, 107 aa, Homo sapiens [sequence omitted]

SEQ ID NO: 12 - protein, 330 aa, Homo sapiens [sequence omitted]

(19) United States
(12) Patent Application Publication
(10) Pub. No.: US 2009/0068196 A1
(43) Pub. Date: Mar. 12, 2009

(54) Pharmaceutical formulation of an antibody against IL13Ralpha1

(76) Inventors: Pierre Goldbach, Rixheim (FR); Hanns-Christian Mahler, Basel (CH)

Correspondence Address: ROCHE PALO ALTO LLC;PATENT LAW DEPT. M/S A2-250, 3431 HILLVIEW AVENUE, PALO ALTO, CA, 94304, US

(21) Appl. No.: 12/217,942

(22) Filed: Jul. 10, 2008

(30) Foreign Application Priority Data
Jul. 10, 2007 (EP) 07112162.8

(51) Int. Cl. A61K 39/395; A61P 11/06

(52) U.S. Cl. 424/158.1

(57) ABSTRACT

Pharmaceutical formulations of an antibody against IL13Rα1 and processes for making the same.

### CROSS REFERENCE TO PRIOR APPLICATIONS

This application claims the benefit of priority under 35 USC §119 to European Application No. EP 07112162.8 filed on Jul. 10, 2007 the contents of which are hereby incorporated in their entirety by reference.

### FIELD OF THE INVENTION

This invention relates to pharmaceutical formulations of an antibody against IL13Rα1, and processes for the preparation and uses of the formulations.

### BACKGROUND OF THE INVENTION

IL-13 is a secreted monomeric peptide produced mainly by Th2 cells but also by mast cells and NK cells. Biological functions of IL-13 include regulation of IgE production and modulation of Th2 development. IL-13 binds to a receptor complex consisting of IL-13 receptor alphal (IL-13Rα1) chain and IL-4 receptor alpha (IL-4Rα) chain. IL-13 binding triggers signal transduction events mainly through STAT6. IL-13 binds with low affinity to the IL-13Rα1 alone and does not bind to IL-4Rα1. Contrary to this, IL-4 binds to IL-4Rα alone and does not bind to IL-13Rα1 alone. Another receptor for IL-13 has been described, the IL-13Rα2. IL-13 binds with high affinity to this receptor. Likely this receptor acts as a decoy receptor.

IL-13 antagonists have been shown effective in animal models for treatment of respiratory indications. For example a soluble mouse IL-13Rα2-IgGFc fusion protein has been used to show efficacy in completely reversing ovalbumin-induced AHR and the number of mucus containing cells. The reversal was obtained even if the treatment is given after full development of the phenotype. In addition, treatment of mice with an IL-13 fusion cytotoxin molecule resulted in reduction of all features of airway disease in a chronic fungal-induced allergic inflammation. In conclusion, IL-13 is a critical mediator of the effector arm of the allergic response.

Antibodies against IL13Rα1 are known from, e.g., WO 96/29417, WO 97/15663, WO 03/080675, Graber et al., Eur. J. Immunol. 28:4286-4298 (1998); Poudrier et al., J. Immunol. 163:1153-1161 (1999); Poudrier et al., Eur. J. Immunol. 30:3157-3164 (2000); Aikawa et al., Cytokine 13:75-84 (2001), and are commercially available from, e.g., R&D Systems Inc. USA.

### SUMMARY OF THE INVENTION

In a first aspect, the invention relates to a pharmaceutical formulation comprising:
1 to 200 mg/mL of an antibody against IL13Rα1;
1 to 100 mM of a buffer;
0.001 to 1% of a surfactant;
(a) 10 to 500 mM of a stabilizer; or
(b) 10 to 500 mM of a stabilizer and 5 to 500 mM of a tonicity agent; or
(c) 5 to 500 mM of a tonicity agent;
at a pH in the range of from 4.0 to 7.0.

In one embodiment the present invention provides a formulation in a liquid form. In another embodiment the present invention provides a formulation in a lyophilized form. In another embodiment the present invention provides a formulation in a liquid form reconstituted from a lyophilized form. Also provided are processes for preparing the subject formulations.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The following definitions are set forth to illustrate and define the meaning and scope of the various terms used to describe the invention herein.

The term “buffer” as used herein denotes a pharmaceutically acceptable excipient, which stabilizes the pH of a pharmaceutical preparation. Suitable buffers are well known in the art and can be found in the literature. Preferred pharmaceutically acceptable buffers comprise but are not limited to histidine-buffers, citrate-buffers, succinate-buffers, acetate-buffers and phosphate-buffers. Still preferred buffers comprise L-histidine or mixtures of L-histidine and L-histidine hydrochloride with pH adjustment with an acid or a base known in the art. The abovementioned buffers are generally used in an amount of about 1 mM to about 100 mM, preferably of about 5 mM to about 50 mM and more preferably of about 10-20 mM. Independently from the buffer used, the pH can be adjusted at a value comprising about 4.0 to about 7.0 and preferably about 5.0 to about 6.5 and still preferably about 5.5 to about 6.0 with an acid or a base known in the art, e.g. hydrochloric acid, acetic acid, phosphoric acid, sulfuric acid and citric acid, sodium hydroxide and potassium hydroxide.

The term “surfactant” as used herein denotes a pharmaceutically acceptable excipient which is used to protect protein formulations against mechanical stresses like agitation and shearing. Examples of pharmaceutically acceptable surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulphate (SDS). Preferred polyoxyethylenesorbitan-fatty acid esters are polysorbate 20, (sold under the trademark Tween 20™) and polysorbate 80 (sold under the trademark Tween 80™). Preferred polyethylene-polypropylene copolymers are those sold under the names Pluronic® F68 or Poloxamer 188™. Preferred Polyoxyethylene alkyl ethers are those sold under the trademark Brij™. Preferred alkylphenolpolyoxyethylene esthers are sold under the tradename Triton-X. When polysorbate 20 (Tween 20™) and polysorbate 80 (Tween 80™) are used they are generally used in a concentration range of about 0.001 to about 1%, preferably of about 0.005 to about 0.1% and more preferably about 0.01% to about 0.04%w/v (weight/volume).

The term “stabilizer” denotes a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland et al. (1993), Crit Rev Ther Drug Carrier Syst 10(4):307-77, Wang (1999) Int J Pharm 185(2):129-88, Wang (2000) Int J Pharm 203(1-2):1-60 and Chi et al. (2003) Pharm Res 20(9):1325-36. Stabilizers include but are not limited to sugars, amino acids, polyols, cyclodextrines, e.g. hydroxypropyl-β-cyclodextrine, sulfobutylethyl-β-cyclodextrin, β-cyclodextrin, polyethylenglycols, e.g. PEG 3000, PEG 3350, PEG 4000, PEG 6000, albumine, human serum albumin (HSA), bovine serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 10 to about 500 mM, preferably in an amount of about 10 to about 300 mM and more preferably in an amount of about 100 mM to about 300 mM.

The term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra- penta- and so forth saccharide. In contrast to polysaccharides the monosaccharides and oligosaccharides are water soluble. Examples of oligosaccharides include sucrose, trehalose, lactose, maltose and raffinose. Preferred sugars are sucrose and trehalose, most preferred is trehalose.

The term “amino acid” as used herein denotes a pharmaceutically acceptable organic molecule possessing an amino moiety located at α-position to a carboxylic group. Examples of amino acids include arginine, glycine, omithine, lysine, histidine, glutamic acid, asparagic acid, isoleucine, leucine, alanine, phenylalanine, tyrosine, tryptophane, methionine, serine, proline. Amino acids are generally used in an amount of about 10 to 500 mM, preferably in an amount of about 10 to about 300 mM and more preferably in an amount of about 100 to about 300 mM

The term “polyols” as used herein denotes pharmaceutically acceptable alcohols with more than one hydroxy group. Suitable polyols comprise to but are not limited to mannitol, sorbitol, glycerine, dextran, glycerol, arabitol, propylene glycol, polyethylene glycol, and combinations thereof. Polyols can be used in an amount of about 10 mM to about 500 mM, preferably in an amount of about 10 to about 300 mM and more preferably in an amount of about 100 to about 300 mM.

A subgroup within the stabilizers are lyoprotectants. The term “lyoprotectant” denotes pharmaceutical acceptable excipients, which protect the labile active ingredient (e.g. a protein) against destabilizing conditions during the lyophilisation process, subsequent storage and reconstitution. Lyoprotectants comprise but are not limited to the group consisting of sugars, polyols (such as e.g. sugar alcohols) and amino acids. Preferred lyoprotectants can be selected from the group consisting of sugars such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine. Lyoprotectants are generally used in an amount of about 10 to 500 mM, preferably in an amount of about 10 to about 300 mM and more preferably in an amount of about 100 to about 300 mM.

A subgroup within the stabilizers are antioxidants. The term “antioxidant” denotes pharmaceutically acceptable excipients, which prevent oxidation of the active pharmaceutical ingredient. Antioxidants comprise but are not limited to ascorbic acid, gluthadion, cysteine, methionine, citric acid, EDTA. Antioxidants can be used in an amount of about 1 to about 100 mM, preferably in an amount of about 5 to about 50 mM and more preferably in an amount of about 5 to about 20 mM.

The term “tonicity agents” as used herein denotes pharmaceutically acceptable tonicity agents. Tonicity agents are used to modulate the tonicity of the formulation. The formulation can be hypotonic, isotonic or hypertonic. Isotonicity in general relates to the osmostic pressure relative of a solution usually relative to that of human blood serum. The formulation according to the invention can be hypotonic, isotonic or hypertonic but will preferably be isotonic. An isotonic formulation is liquid or liquid reconstituted from a solid form, e.g. from a lyophilised form and denotes a solution having the same tonicity as some other solution with which it is compared, such as physiologic salt solution and the blood serum. Suitable tonicity agents comprise but are not limited to sodium chloride, potassium chloride, glycerine and any component from the group of amino acids, sugars, in particular glucose. Tonicity agents are generally used in an amount of about 5 mM to about 500 mM. In a preferred formulation the amount of tonicity agent is is in the range of 50 mM to 300 mM.

Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethylenglycols and salts. An example for a sugar which can at the same time be a stabilizer and a tonicity agent is trehalose.

The compositions may also contain “adjuvants” such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol, sorbic acid, and the like. Preservatives are generally used in an amount of about 0.001 to about 2%(w/v). Preservatives comprise but are not limited to ethanol, benzyl alcohol, phenol, m-cresol, p-chlor-m-cresol, methyl or propyl parabens, benzalkonium chloride.

The term “liquid” as used herein in connection with the formulation according to the invention denotes a formulation which is liquid at a temperature of at least about 2 to about 8° C. under atmospheric pressure.

The term “lyophilizate” as used herein in connection with the formulation according to the invention denotes a formulation which is manufactured by freeze-drying methods known in the art per se. The solvent (e.g. water) is removed by freezing following sublimation under vacuum and desorption of residual water at elevated temperature. The lyophilisate has usually a residual moisture of about 0.1 to 5% (w/w) and is present as a powder or a physical stable cake. The lyophilizate is characterized by a fast dissolution after addition of a reconstitution medium.

The term “reconstituted formulation” as used herein in connection with the formulation according to the invention denotes a formulation which is lyophilized and redissolved by addition of reconstitution medium. The reconstitution medium comprise but is not limited to water for injection (WFI), bacteriostatic water for injection (BWFI), sodium chloride solutions (e.g. 0.9% (w/v) NaCl), glucose solutions (e.g. 5% glucose), surfactant, containing solutions (e.g. 0.01% polysorbate 20), a pH -buffered solution (eg. phosphate-buffered solutions).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

### Formulations

Exemplary antibodies against IL13Rα1 usable in the formulations of the invention are described in WO2006/072564 and US20060263356, the disclosures of which are incorporated herein by reference in their entirety. Such exemplary antibodies include antibodies which are characterized in comprising as heavy chain complementarity determining regions (CDRs) of SEQ ID NO: 1 and as light chain CDRs the CDRs of SEQ ID NO:2; as heavy chain CDRs the CDRs of SEQ ID NO:3 and as light chain CDRs the CDRs of SEQ ID NO:4; as heavy chain CDRs the CDRs of SEQ ID NO:5 and as light chain CDRs the CDRs of SEQ ID NO:6; as heavy chain CDRs the CDRs of SEQ ID NO:7 and as light chain CDRs the CDRs of SEQ ID NO:8; or as heavy chain CDRs the CDRs of SEQ ID NO:9 and as light chain CDRs the CDRs of SEQ ID NO:10.

The CDR sequences can be determined according to the standard definition of Kabat et al., Sequences of Proteins of Immunological Interest, 5th ed., Public Health Service, National Institutes of Health, Bethesda, Md. (1991). On this basis, the complementarity determining regions (CDRs) have the following sequences:

Heavy chain CDRs:
CDR1 (aa 31-35) of SEQ ID NO: 1, 3, 5, 7, 9,
CDR2 (aa 50-66) of SEQ ID NO: 1, 3, 5, 7, 9,
CDR3 (aa 99-108) of SEQ ID NO: 1, 3, 9,
CDR3 (aa 99-107) of SEQ ID NO: 5,
CDR3 (aa 99-112) of SEQ ID NO: 7;
   Light chain CDRs:
CDR1 (aa 24-34 ) of SEQ ID NO: 2, 4, 6, 10,
CDR1 (aa 24-35 )of SEQ ID NO: 8,
CDR2 (aa 50-56) of SEQ ID NO: 2, 4, 6, 10,
CDR2 (aa 51-57) of SEQ ID NO:8 and
CDR3 (aa 89-97) of SEQ ID NO: 2, 4, 6, 10,
CDR3 (aa 90-97) of SEQ ID NO: 8.

Preferred antibodies are characterized in comprising:
a) as heavy chain variable region SEQ ID NO:1, as light chain variable region SEQ ID NO:2, as K light chain constant region SEQ ID NO:11 and as γ1 heavy chain constant region SEQ ID NO:12 optionally with mutations L234A and L235A or D265A and N297A,
b) as heavy chain variable region SEQ ID NO:3 and as light chain variable region of SEQ ID NO:4, as κ light chain constant region SEQ ID NO:11 and as γ1 heavy chain constant region SEQ ID NO:12 optionally with mutations L234A and L235A or D265A and N297A,
c) as heavy chain variable region SEQ ID NO:5 and as light chain variable region SEQ ID NO:6, as κ light chain constant region SEQ ID NO:11 and as γ1 heavy chain constant region SEQ ID NO:12 optionally with mutations L234A and L235A or D265A and N297A,
d) as heavy chain variable region SEQ ID NO:7 and as light chain variable region SEQ ID NO:8, as κ light chain constant region SEQ ID NO:11 and as γ1 heavy chain constant region SEQ ID NO:12 optionally with mutations L234A and L235A or D265A and N297A, or
e) as heavy variable region SEQ ID NO:9 and as light chain variable region SEQ ID NO:10, as κ light chain constant region SEQ ID NO:11 and as γ1 heavy chain constant region SEQ ID NO:12 optionally with mutations L234A and L235A or D265A and N297A.

In one embodiment the antibody is characterized in binding to IL-13Rα1 in competition to antibody LC5002-002, LC5002-003, LC5002-005, LC5002-007 and/or LC5002-018. Preferably the antibody is characterized in comprising as variable regions the variable regions of LC5002-002, LC5002-003, LC5002-005, LC5002-007 or LC5002-018. The variable regions of these antibodies are shown in SEQ ID NO: 1-10. Useful constant regions are well known in the state of the art. Examples are shown as SEQ ID NO: 11-12 in table 1.

TABLE 1

| | |
|---|---|
| SEQ ID NO: 1 | heavy chain variable domain of HuMab LC5002-002 |
| SEQ ID NO: 2 | light chain variable domain of HuMab LC5002-002 |
| SEQ ID NO: 3 | heavy chain variable domain of HuMab LC5002-003 |
| SEQ ID NO: 4 | light chain variable domain of HuMab LC5002-003 |
| SEQ ID NO: 5 | heavy chain variable domain of HuMab LC5002-005 |
| SEQ ID NO: 6 | light chain variable domain of HuMab LC5002-005 |
| SEQ ID NO: 7 | heavy chain variable domain of HuMab LC5002-007 |
| SEQ ID NO: 8 | light chain variable domain of HuMab LC5002-007 |
| SEQ ID NO: 9 | heavy chain variable domain of HuMab LC5002-018 |
| SEQ ID NO: 10 | light chain variable domain of HuMab LC5002-018 |
| SEQ ID NO: 11 | κ light chain constant region |
| SEQ ID NO: 12 | γ1 heavy chain constant region |

In a preferred embodiment of the invention the antibody contains a human γ1 heavy chain comprising:
a) amino acid sequence Pro₂₃₃Val₂₃₄Ala₂₃₅ with deletion of Gly₂₃₆ and/or amino acid sequence Gly₃₂₇Leu₃₂₈Pro₃₂₉Ser₃₃₀Ser₃₃₁,
b) amino acid sequence Ala₂₃₄Ala₂₃₅ or
c) amino acids Ala₂₆₅ and Ala₂₉₇.

In one embodiment the present invention provides a formulation wherein the antibody is present in an amount in the range of from 10 to 150 mg/mL, preferably from 10 to 50 mg/mL.

The antagonistic monoclonal antibodies against IL-13Rα1 may be produced by hybridoma cell lines. The preferred hybridoma cell lines are (hu-MAB<h-IL-13R alpha>LC.5002-002 (DSM ACC2709), hu-MAB<h-IL-13Ralpha>LC.5002-003 (DSM ACC2710), hu-MAB<h-IL-13Ralpha>LC.5002-005 (DSM ACC2711), hu-MAB<h-IL-13R alpha>LC.5002-007 (DSM ACC2712)) which were deposited on 13.01.2005 with Deutsche Sammlung von Mikroorganismen und Zellkulturen GmbH (DSMZ), Germany.

The antibodies useful in the formulations according to the invention are preferably produced by recombinant means, e.g. by those described in WO2006/072564. Such methods are widely known in the state of the art and comprise protein expression in prokaryotic and eukaryotic cells with subsequent isolation of the antibody polypeptide and usually purification to a pharmaceutically acceptable purity. For the protein expression, nucleic acids encoding light and heavy chains or fragments thereof are inserted into expression vectors by standard methods. Expression is performed in appropriate prokaryotic or eukaryotic host cells like CHO cells, NS0 cells, SP2/0 cells, HEK293 cells, COS cells, yeast, or E. coli cells, and the antibody is recovered from the cells (supernatant or cells after lysis) by standard techniques, including alkaline/SDS treatment, CsCl banding, column chromatography, agars gel electrophoresis, and others well known in the art, e.g. as described in WO2006/072564.

In one preferred embodiment the invention provides a liquid formulation which comprises:
1 to 50 mg/mL huMAb-IL-13Rα1,
20 mM L-histidine HCl,
240 mM trehalose,
0.02% polysorbate 20,
at pH 6.0.

In another preferred embodiment the invention provides a liquid formulation which comprises:
1 to 50 mg/mL huMAb-IL-13Rα1,
20 mM L-histidine HCl,
240 mM trehalose,
0.04% polysorbate 20,
at pH 6.0.

In another preferred embodiment the invention provides a liquid formulation which comprises:
1 to 50 mg/mL huMAb-IL-13Rα1,
20 mM L-histidine HCl,
160 mM trehalose,
100 mM glycine
0.02% polysorbate 20,
at pH 6.0.

In another preferred embodiment the invention provides a liquid formulation which comprises:
1 to 50 mg/mL huMAb-IL-13Rα1,
20 mM Na acetate
240 mM trehalose,
0.02% polysorbate 20,
at pH 5.5.

In another preferred embodiment the invention provides a liquid formulation which comprises:
1 to 50 mg/mL huMAb-IL-13Rα1,
20 mM Na acetate
240 mM trehalose,
0.04% polysorbate 20,
at pH 5.5.

In another preferred embodiment the invention provides a liquid formulation which comprises:
1 to 50 mg/mL huMAb-IL-13Rα1,
20 mM Na succinate
240 mM trehalose,
0.02% polysorbate 20,
at pH5.5.

In another preferred embodiment the invention provides a liquid formulation which comprises:
1 to 50 mg/mL huMAb-IL-13Rα1,
20 mM L-histidine HCl,
240 mM trehalose,
0.04% polysorbate 20,
at pH 6.0.

The formulations according to the invention have new and inventive properties causing a benefit for a patient suffering from asthma or an allergic disease.

The invention further comprises the use of a formulation according to the invention for the manufacture of a medicament for asthma treatment.

A composition of the present invention can be administered by a variety of methods known in the art. As will be appreciated by the skilled artisan, the route and/or mode of administration will vary depending upon the desired results.

To administer a composition of the invention by certain routes of administration, it may be necessary to dilute the composition in a diluent. Pharmaceutically acceptable diluents include saline, glucose, Ringer and aqueous buffer solutions.

The composition must be sterile and fluid to the extent that the composition is deliverable by syringe. In addition to water, the carrier can be an isotonic buffered saline solution, ethanol, polyol (e.g., glycerol, propylene glycol, and liquid polyetheylene glycol, and the like), and suitable mixtures thereof.

The formulation according to the invention can be administered by intravenous (i.v.), subcutaneous (s.c.) or any other parental administration means such as those known in the pharmaceutical art.

The formulation according to the invention can be prepared by methods known in the art, e.g. ultrafiltration-diafiltration, dialysis, addition and mixing, lyophilisation, reconstitution, and combinations thereof. Examples of preparations of formulations according to the invention can be found hereinafter.

### EXAMPLES

Examples of the formulations encompassed by the present invention and within the scope of the invention are provided in the following examples. These examples are provided to enable those skilled in the art to more clearly understand and to practice the present invention. They should not be considered as limiting the scope of the invention, but merely as being illustrative and representative thereof.

### Example 1

### Preparation of Liquid Formulations

huMAb-IL13-Rα1 prepared and obtained as described in WO2006/072564 and US20060263356 was provided at a concentration of approximately 10 to 15 mg/mL in a 20 mM histidine buffer at a pH of approximately 6.0.

For the preparation of the liquid formulations huMAb-IL13-Rα1 was buffer-exchanged against a diafiltration buffer containing the anticipated buffer composition and concentrated by ultrafiltration to an antibody concentration of approximately 15 mg/mL. After completion of the ultrafiltration operation, the excipients (e.g. trehalose) were added as 2-10-fold stock solutions to the antibody solution. The surfactant was then added as a 100 to 200-fold stock solution. Finally the protein concentration was adjusted with a buffer to the final huMAb-EL13-Rα1 concentration of approx. 10 mg/mL.

All formulations were sterile-filtered through 0.22 μm low protein binding filters and aseptically filled under nitrogen atmosphere into sterile 6 mL glass vials closed with ETFE (Copolymer of ethylene and tetrafluoroethylene)-coated rubber stoppers and alucrimp caps. The fill volume was approx. 2.4 mL. These formulations were stored at different climate conditions (5° C., 25° C. and 40° C.) for different intervals of time and stressed by shaking (1 week at a shaking frequency of 200 min⁻¹ at 5° C.) and freeze-thaw stress methods. The samples were analyzed before and after applying the stress tests by the analytical methods 1) UV spectrophotometry, and 2) Size Exclusion Chromatography (SEC).

Size Exclusion Chromatography (SEC) was used to detect soluble high molecular weight (HMW) species (aggregates) and low molecular weight hydrolysis products (LMW) in the formulations. The method was performed on a Water Alliance 2795 HPLC instrument equipped with a Tosohaas TSK G3000 SWXL column. Intact monomer, aggregates and hydrolysis products were separated by an isocratic elution profile, using 0.2M K₂HPO₄/0.25M KCL, pH 7.0 as mobile phase, and were detected at a wavelength of 220 nm. UV spectroscopy, used for determination of protein content, was performed on a Varian Cary Bio UV spectrophotometer in a wavelength range from 240 nm to 400 nm. Neat protein samples were diluted to approx. 0.5 mg/mL with the corresponding formulation buffer. The protein concentration was calculated according to equation 1.

$$Protein content = \frac{A ( 280 ) - A ( 320 ) \times dil . factor}{ɛ 〈 {cm}^{2} \text{/} mg 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient ε. Table 2 illustrates protein concentration and HPLC size exclusion data for several formulations.

TABLE 2

| | Protein conc. | Size Exclusion-HPLC | | |
|---|---|---|---|---|
| Timepoint | (mg/mL) | HMW (%) | Monomer (%) | LMW (%) |

Formulation A
Storage at 2-8° C.
10 mg/mL huMAb-IL-13Rα1,
20 mM L-histidine HCl,
240 mM trehalose,
0.02% polysorbate 20,
at pH 6.0

| | | | | |
|---|---|---|---|---|
| Initial | 10.3 | 1.9 | 98.0 | 0.1 |
| 2 weeks | 11.1 | 1.9 | 98.0 | 0.1 |
| 4 weeks | 10.8 | 2.2 | 97.7 | 0.2 |
| 17 weeks | 10.3 | 2.1 | 97.8 | 0.2 |

Formulation B
Storage 2-8° C.
10 mg/mL huMAb-IL-13Rα1,
20 mM L-histidine HCl,
240 mM trehalose,
0.04% polysorbate 20,
at pH 6.0

| | | | | |
|---|---|---|---|---|
| Initial | 10.2 | 1.9 | 98.0 | 0.1 |
| 2 weeks | 11.1 | 1.9 | 98.0 | 0.1 |
| 4 weeks | 10.3 | 2.2 | 97.6 | 0.2 |
| 17 weeks | 10.5 | 2.3 | 97.6 | 0.2 |

Formulation C
Storage at 2-8° C.
10 mg/mL huMAb-IL-13Rα1,
20 mM L-histidine HCl,
160 mM trehalose,
100 mM glycine
0.02% polysorbate 20,
at pH 6.0

| | | | | |
|---|---|---|---|---|
| Initial | 10.2 | 1.9 | 98.0 | 0.1 |
| 2 weeks | 10.3 | 1.9 | 98.0 | 0.1 |
| 4 weeks | 9.9 | 2.2 | 97.7 | 0.2 |
| 17 weeks | 10.5 | 2.1 | 97.7 | 0.2 |

Formulation D
Storage at 2-8° C.
10 mg/mL huMAb-IL-13Rα1,
20 mM Na acetate
240 mM trehalose,
0.02% polysorbate 20,
at pH 5.5

| | | | | |
|---|---|---|---|---|
| Initial | 9.9 | 1.0 | 98.7 | 0.3 |
| 2 weeks | 10.4 | 1.0 | 98.7 | 0.3 |
| 5 weeks | 10.4 | 1.0 | 98.7 | 0.3 |
| 12 weeks | 10.6 | 1.4 | 98.2 | 0.4 |

Formulation E
Storage at 2-8° C.
10 mg/mL huMAb-IL-13Rα1,
20 mM Na acetate
240 mM trehalose,
0.04% polysorbate 20,
at pH 5.5

| | | | | |
|---|---|---|---|---|
| Initial | 10.0 | 2.1 | 97.8 | 0.1 |
| 2 weeks | 10.2 | 2.1 | 97.9 | 0.1 |
| 4 weeks | 10.3 | 2.2 | 97.7 | 0.1 |
| 17 weeks | 9.9 | 2.1 | 97.8 | 0.2 |

Formulation F
Storage at 2-8° C.
10 mg/mL huMAb-IL-13Rα1,
20 mM Na succinate
240 mM trehalose,
0.02% polysorbate 20,
at pH 5.5

| | | | | |
|---|---|---|---|---|
| Initial | 10.2 | 2.6 | 97.2 | 0.1 |
| 2 weeks | 10.3 | 2.7 | 97.2 | 0.1 |
| 4 weeks | 10.4 | 3.0 | 96.9 | 0.1 |
| 17 weeks | 10.3 | 2.6 | 97.2 | 0.2 |

Formulation G
Storage at 2-8° C.
10 mg/mL huMAb-IL-13Rα1,
20 mM Na succinate
240 mM trehalose,
0.04% polysorbate 20,
at pH 5.5

| | | | | |
|---|---|---|---|---|
| Initial | 10.3 | 2.7 | 97.2 | 0.1 |
| 2 weeks | 10.7 | 2.7 | 97.2 | 0.1 |
| 4 weeks | 10.7 | 2.9 | 97.0 | 0.1 |
| 17 weeks | 10.1 | 2.7 | 97.1 | 0.2 |

### Example 2: Preparation of Lyophilized Formulations and Liquid Formulations Reconstituted from Lyophilized Formulations

Solutions of approx. 10 mg/ml huMAb-IL13-Rα1 were prepared as described in Example 2 and lyophilized using the freeze-drying cycle reported in Table 3.

TABLE 3
Freeze-drying Cycle type I

| | | | | Vacuum |
|---|---|---|---|---|
| | Shelf temperature | Ramp Rate | Hold time | Set point |
| Step | (° C.) | (° C./min) | (min) | (μbar) |
| Pre-cooling | 5° C. | 0.0 | 60 | — |
| Freezing | −40° C. | 1.0 | 150 | — |
| Primary Drying | −25° C. | 0.5 | 3660 | 80 |
| Secondary | +25° C. | 0.2 | 300 | 80 |
| Drying | | | | |

The product was first cooled from room temperature to approx 5° C. (pre-cooling), followed by a freezing step at −40° C. with a plate cooling rate of approx. 1° C./min, followed by a holding step at −40° C. for about 2 hours. The first drying step was performed at a plate temperature of approx. −25° C. and a chamber pressure of approx. 80 μbar for about 62 hours. Subsequently, the second drying step started with a temperature ramp of 0.2° C./min from −25° C. to 25° C., followed by a holding step at 25° C. for at least 5 hours at a chamber pressure of approx. 80 μbar.

Lyophilization was carried out in an Usifroid SMH-90 LN2 freeze-dryer (Usifroid, Maurepas, France). All lyophilized cakes had a residual water content of about 0.1 to 2.0% as determined by the Karl-Fischer method. The freeze-dried samples were incubated at different temperatures for different intervals of time.

The lyophilized formulations were reconstituted to a final volume of 2.4 mL with water for injection (WFD yielding an isotonic formulation with an antibody concentration of approx. 10 mg/mL. The reconstitution time of the freeze-dried cakes was below 1 min. Analysis of the reconstituted samples was either performed immediately after reconstitution, or after a 24 hour incubation period of the reconstituted liquid sample at 25° C.

The samples were analyzed by 1) UV spectrophotometry and 2) Size Exclusion Chromatography (SEC). Table 4 illustrates protein concentration and HPLC size exclusion data for several formulations.

TABLE 4

| | |
|---|---|
| Formulation H | 10 mg/mL huMAb-IL-13Rα1, |
| Storage at 2-8° C. | 20 mM L-histidine HCl, |
| | 240 mM trehalose, |
| | 0.02% polysorbate 20, |
| | at pH 6.0 |

| | Protein conc. | Size Exclusion - HPLC | | |
|---|---|---|---|---|
| Timepoint | (mg/mL) | HMW (%) | Monomer (%) | LMW (%) |
| Initial | 10.4 | 1.9 | 98.0 | 0.1 |
| 2 weeks | 10.6 | 1.9 | 98.0 | 0.1 |
| 5 weeks | 10.4 | 2.2 | 97.7 | 0.2 |
| 12 weeks | 10.2 | 2.1 | 97.8 | 0.2 |

| | |
|---|---|
| Formulation I | 10 mg/mL huMAb-IL-13Rα1, |
| Storage at 2-8° C. | 20 mM L-histidine HCl, |
| | 240 mM trehalose, |
| | 0.04% polysorbate 20, |
| | at pH 6.0 |

| | Protein conc. | Size Exclusion - HPLC | | |
|---|---|---|---|---|
| Timepoint | (mg/mL) | HMW (%) | Monomer (%) | LMW (%) |
| Initial | 10.3 | 0.9 | 98.8 | 0.3 |
| 2 weeks | 10.0 | 0.9 | 98.8 | 0.3 |
| 4 weeks | 10.8 | 0.9 | 98.8 | 0.3 |
| 12 weeks | 10.4 | 1.0 | 98.6 | 0.4 |

| | |
|---|---|
| Formulation J | 10 mg/mL huMAb-IL-13Rα1, |
| Storage at 2-8° C. | 20 mM Na succinate, |
| | 240 mM trehalose, |
| | 0.02% polysorbate 20, |
| | at pH 6.0 |

| | Protein conc. | Size Exclusion - HPLC | | |
|---|---|---|---|---|
| Timepoint | (mg/mL) | HMW (%) | Monomer (%) | LMW (%) |
| Initial | 10.4 | 2.7 | 97.1 | 0.1 |
| 2 weeks | 10.8 | 2.6 | 97.5 | n.d. |
| 4 weeks | 10.7 | 2.9 | 97.0 | 0.1 |
| 17 weeks | 10.1 | 2.8 | 97.1 | 0.2 |

| | |
|---|---|
| Formulation K | 10 mg/mL huMAb-IL-13Rα1, |
| Storage at 2-8° C. | 20 mM Na succinate, |
| | 240 mM trehalose, |
| | 0.04% polysorbate 20, |
| | at pH 6.0 |

| | Protein conc. | Size Exclusion - HPLC | | |
|---|---|---|---|---|
| Timepoint | (mg/mL) | HMW (%) | Monomer (%) | LMW (%) |
| Initial | 10.2 | 2.8 | 97.1 | 0.1 |
| 2 weeks | 11.0 | 2.6 | 97.4 | n.d. |
| 4 weeks | 10.1 | 3.1 | 96.8 | 0.1 |
| 17 weeks | 9.9 | 2.8 | 97.1 | 0.2 |

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A pharmaceutical formulation comprising:
1 to 200 mg/mL of an antibody;
1 to 100 mM of a buffer;
0.001 to 1% of a surfactant;
(a) 10 to 500 mM of a stabilizer; or
(b) 10 to 500 mM of a stabilizer and 5 to 500 mM of a tonicity agent; or
(c) 5 to 500 mM of a tonicity agent;
at a pH in the range of from 4.0 to 7.0,
wherein the antibody is an antibody against IL13Rα1.

2. The formulation according to claim 1 which is a liquid formulation or a lyophilized formulation or a liquid formulation reconstituted from a lyophilized formulation.

3. The formulation according to claim 1, wherein the antibody concentration is in the range of 10 mg/mL to 150 mg/ml.

4. The formulation according to claim 1, wherein the stabilizer is trehalose.

5. The formulation according to claim 1, wherein the surfactant is polysorbate.

6. The formulation according to claim 1, wherein the buffer is a histidine-buffer.

7. The formulation according to claim 1, which comprises a tonicity agent.

8. The formulation according to claim 1, wherein the tonicity agent is trehalose.

9. The lyophilized formulation according to claim 1, comprising:
1 to about 50 mg/mL huMAb-IL-13Rα1,
20 mM L-histidine HCl,
240 mM trehalose,
0.02% polysorbate 20,
at pH 6.0.

10. The lyophilized formulation according to claim 1, comprising:
1 to 50 mg/mL huMAb-IL-13Rα1,
20 mM L-histidine HCl,
240 mM trehalose,
0.04% polysorbate 20,
at pH 6.0.

11. The lyophilized formulation according to claim 1, comprising:
1 to 50 mg/mL huMAb-IL-13Rα1,
20 mM Na succinate
240 mM trehalose,
0.02% polysorbate 20,
at pH 5.5

12. The lyophilized formulation according to claim 1, comprising:
1 to 50 mg/mL huMAb-IL-13Rα1,
20 mM L-histidine HCl,
240 mM trehalose,
0.04% polysorbate 20,
at pH 6.0.

13. A method of treating asthma or allergy, the method comprising administering to a patient in need thereof an effective amount of a formulation of claim 1.
